(19) United States
(12) Patent Application Publication
(10) Pub. No.: US 2024/0198055 A1
(43) Pub. Date: Jun. 20, 2024

(54) APPARATUS AND METHOD FOR SECUREMENT OF A FLEXIBLE CONDUIT

(71) Applicant: JAVELO HEALTH LIMITED, Edinburgh (GB)

(72) Inventors: Ashton BARNETT-VANES, Edinburgh (GB); Alan KITCHING, Edinburgh (GB)

(21) Appl. No.: 18/288,130

(22) PCT Filed: Apr. 28, 2022
(86) PCT No.: PCT/GB2022/051089

§ 371 Date: Oct. 24, 2023

(30) Foreign Application Priority Data
Apr. 30, 2021 (GB) 2106271.6

(51) Int. Cl. A61M 25/02

(52) U.S. Cl. CPC A61M 25/02; A61M 2025/0206; A61M 2025/026; A61M 2205/584; A61M 2205/587

(57) ABSTRACT

Disclosed is a securement device (100) for securement of a flexible conduit (101a, 101b), a medical system comprising a securement device and a conduit secured thereto; and a method of securing a flexible conduit. A securement device comprises a support portion and elastically extendable first (132a) and second straps (120) coupled thereto. In use, the straps are attached to a flexible conduit, which is urged into a curved or convoluted pathway. The straps may elastically extend when tension is applied to the conduit and the securement device isolates the conduit extending to one side of the securement device from tension applied to the conduit on the other side of the securement device.

### FIELD OF THE INVENTION

The invention relates to the field of fluid line attachment in a clinical setting, in particular the attachment of catheters, intravascular lines and the like to a patient in a clinical setting.

### BACKGROUND TO THE INVENTION

Medical treatments commonly involve infusing fluids into or withdrawing fluids from a patient, by connecting a fluid line to a body cavity such as the thorax, abdomen, cranium or luminal conduit such as a vein, artery, spinal cord, urethra, bowel etc.

For example, an intravenous (IV) line may be used to deliver drugs, deliver or exchange plasma or to hydrate a patient, whereas bodily fluids may be removed via urinary or enteric catheterisation, or surgical drains may be required during or after surgery.

A patient or body part can remain relatively mobile while a fluid line is attached and a patient may forget or be unaware of the fluid line, particularly for lengthy treatments or when regaining consciousness. A clinical setting may also be crowded with additional fluid lines and medical apparatus and clinicians may need to frequently work in close proximity to fluid lines. Accordingly, unplanned removal of lines from a patient is a common problem and can lower the quality of care and may lead to adverse clinical outcomes.

Unintended line removal of fluid lines delivering even low cost drugs or fluids can lead to wastage, extend treatment time and cause distress to a patient. It is also known for dislodged lines to leave medical apparatus, such as a needle, in the patient which may require imaging or surgical intervention to remove. Furthermore, the consequence of unintended line removal in some settings can be severe or even fatal, particularly in emergency or surgical settings.

A number of approaches to secure fluid lines and isolate the entry point from forces applied to a line are commonly used.

Adhesive or tape can be used to secure a fluid line. Although this approach can be readily adapted to different types or diameters of line, hair, sweat or other fluids can weaken the adhesion to a patient's skin and at best only limited pull resistance can be achieved. These measures must be constantly renewed and, over time, adhesives and adhesive tapes can cause skin irritation and, in some instances pain or injury may result if removed by pulling on a fluid line.

Devices are known which sever a fluid line or disconnect a bespoke in-line connector, however in addition to cost, treatment is interrupted and clinician time is taken to re-establish a connection.

It is also know to use a strap around a patient's limb or other body part and attach a fluid line to the strap. One such solution is the Stedline™ sleeve (manufactured by Redpoint International, Vancouver, Washington, USA). An IV line is secured to a wrist band by webbing straps provided with hook and loop fastening surfaces. Each strap is secured to the band by a button, around which the IV line can be wrapped. The convoluted path is relatively effective at isolating line tension applied proximally (i.e. between the band and the fluid source) to the band to the portion of the line distal to the band (i.e. between the band and the body entry point). However, securing an IV line to the Stedline is relatively time consuming, and the device only provides the options of attaching a line straight through the strap (which offers very limited isolation of tension) or via a complete loop. Accordingly, the device is prone to causing kinks or folds in an IV line (due to too great a curvature) and so is suitable only for a limited range of applications.

U.S. Pat. No. 4,445,894 is another securement device that utilises a band and straps provided with hook and loop fastenings to accommodate a range of fluid line sizes. The device includes fabric straps that extend in opposite directions away from a stitching line, which can be wrapped around a fluid line. The amount of tension that can be applied to a fluid line is limited, however, because the opposed straps act to twist a fluid line into alignment with the straps.

Devices are also known in which the fluid line is secured or clipped to a rigid or semi rigid form, for example as shown in U.S. Pat. No. 5,916,199, WO2020124214, or the IV-bracelet manufactured by Meadow Health Care, Lund, Sweden; or a series of clips, such as shown in EP 0916361 or WO2015079333. A clip inherently limits the number and range of line diameters that may be secured, and the point at which a fluid line exits a rigid form can focus pressure and lead to kinking and blockage of a fluid line.

Accordingly there remains a need to address one or more of the deficiencies encountered with existing fluid line securements.

### SUMMARY OF THE INVENTION

According to an aspect of the invention there is provided a conduit securement device, for securing a flexible conduit, the device comprising:
a support portion;
an elastically extendable first strap coupled to the support portion; a portion of the first strap being configured to be attached around a first region of a conduit;
an elastically extendable second strap coupled to the support portion; a portion of the second strap being configured to be attached around a longitudinally adjacent second region of said conduit; and
a tertiary attachment for attaching a further region of said flexible conduit to the support portion said further region being longitudinally adjacent the second region;
wherein, when the first and second straps and the tertiary attachment are attached to longitudinally adjacent first, second and tertiary regions of a flexible conduit and secure the flexible conduit to the securement device, the flexible conduit is urged into a curved or convoluted pathway by the straps;
wherein, tension applied longitudinally to a conduit secured to the device acts to straighten the curved or convoluted pathway and extend or further extend the first and/or second strap and increase a static friction with the conduit.

In use, longitudinally adjacent portions of a flexible conduit are attached to the first and second straps and the tertiary attachment, to secure the conduit to the device. The first and second straps may be attached around (e.g. wrapped around) the flexible conduit, such as a fluid line or an electrical or optical conduit. The conduit is urged to a curved or convoluted pathway across the device.

The curved or convoluted pathway and static friction between the straps and the conduit provides a degree of “pull resistance” i.e. to resist longitudinal movement of the conduit in relation to the securement device.

Tension applied to the conduit longitudinally (i.e. wherein at least a component of force is applied along a length of the conduit), proximal or distal to the securement device, acts to straighten the pathway. Additionally, as the pathway straightens, the first and/or second strap elastically extend (or further elastically extend), and increase the static friction with the conduit, resisting longitudinal displacement of the conduit. In effect, the securement device isolates the conduit extending distally from the device from forces applied between the securement device and the conduit extending proximally from the device, and vice versa. The securement device thereby reduces the risk that the entry point of the conduit or associated medical apparatus to a patient is disrupted.

Furthermore, the elastically deformable straps can deform to accommodate lateral movement or bending of the conduit proximal or distal to the device, reducing the risk of bending or crimping of the conduit.

In use, adjacent first, second any tertiary regions of a flexible conduit attached to a strap or tertiary attachment may be directly adjacent along a length of the conduit, such that a continuous length of the conduit is attached, or may be spaced apart from one another such that the first second and/or tertiary regions may be separated by lengths of conduit that are not attached to a strap or attachment.

When the conduit is secured to the securement device, the first strap may be elastically extended to apply a first force to the flexible conduit, and/or the second strap may be elastically extended to apply a second force to the flexible conduit. In some embodiments, the conduit may be urged to follow the curved or convoluted pathway manually, at least in part, when securing the conduit to the securement device.

At least a component of the first and second forces are applied normal to the longitudinal direction of the conduit. At least a component of the first and second forces are applied normal to the longitudinal direction of the conduit, in opposite directions in relation to a longitudinal direction of the conduit.

It will be understood that a longitudinal direction of a flexible conduit may, at least to some extent, vary where the conduit extends along a curved or convoluted pathway.

When a longitudinal force is applied along the conduit (proximal to or distal to the securement device), the first and/or the second strap may elastically extend (or further extend) and apply a corresponding first and/or second force to the flexible conduit. At least a component of the second force may oppose the first force. At least a component of the second force may oppose the first force, in relation to a longitudinal direction of the conduit.

Where we refer to a longitudinal force applied to the conduit, we refer to a force applied to the conduit in relation to the securement device (i.e. as might occur through movement of either), at least a component of which is parallel to the conduit.

Where the first and second straps elastically extend, at least a component of the second force may oppose the first force.

The first and second forces urge the conduit and the securement device back towards an “at-rest” or relaxed configuration (i.e. in which minimal or no external forces are applied to the conduit). In the relaxed configuration, the first and second forces may balance one another, or the first and/or second force may balance a resilient bias of the conduit towards a straight, or straighter configuration. In some embodiments, in the relaxed configuration, the first and/or second band may not be elastically extended.

It will be understood that the first and second forces are forces applied to a flexible conduit resulting from elastic deformation of the straps. Furthermore, by “opposes” we mean that a force or component thereof is in an opposite direction, i.e. acting parallel to but in the opposite sense, to another force. Two such forces may be entirely opposed, i.e. parallel and in opposite directions to one another, or two such forces may be at an angle to one another, but not at an angle orthogonal or greater to one another. Preferably the forces are at an acute angle to one another, for example less than around 45 degrees, 40 degrees, 35 degrees or 30 degrees. It will be further understood that the direction of first or second forces applied along first or second regions of a flexible conduit may vary, where the straps flex or elastically deform to the curved or convoluted pathway.

At least a portion of the first strap and the second strap may be elastically extendable. In some embodiments, all of each of the straps is elastically extendable.

The portions of the first and second straps attachable around a conduit may be attachable entirely around a conduit, for example to form a loop defining a closed channel; or at least partly around a conduit, for example provided with means defining an open channel, configured to cup or grip a conduit.

The portions of the first and second straps attachable around a conduit are advantageously flexible or elastically deformable. Attachment around a conduit via a flexible or elastically deformable portion assists in evenly distributing forces and in use can allow the attached portion to conform to a curvature of the conduit and/or changes in the curvature of the conduit as might be caused by movement of a patient or the conduit.

The first and second straps may be releasably attachable around a conduit. A releasable attachment provides for securement to and release of a conduit from the device to be simply and quickly achieved.

In some embodiments the portions of the first and/or second straps attachable around a conduit are freely movable in relation to the support portion. In use, movement of the conduit with respect to the securement device in a wide range of directions can thereby be accommodated, as the distance and position of the attached portions of the straps in relation to the support portion can be varied. This arrangement may further reduce the risk of crimping or folding a conduit.

The first strap may comprise a first free end. The first free end may be attachable around a conduit. The second strap may comprise a second free end. The second free end may be attachable around a conduit.

The first and/or second strap may be configured to wrap around a conduit. The first and/or second free end may be couplable to another portion of the corresponding strap or the support portion, to define a corresponding first or second loop.

The elastically deformable straps, in particular embodiments configured to wrap around a conduit, may accommodate a range of conduit diameters and/or multiple conduits.

The first and second loops may be elastically deformable.

The securement device may be configured such that adjacent first and second loops may wrap around a conduit in opposite directions, and optionally opposite senses.

By “sense” of a loop, we refer to the clockwise or anticlockwise direction that the loop curves along the strap towards a free end thereof, as viewed along a longitudinal direction.

Each strap may include a fixed portion, such as a fixed end, coupled to the support portion; an intermediate portion and a free end.

At least an intermediate portion of the first and/or second strap may be elastically extendable. In some embodiments other portions, or all of said strap(s) may be elastically extendable. For example at least a portion of each strap may be formed from an elastic material, such as an elastomer or an elastic fabric.

In some embodiments, the securement device comprises a first strap having a first fixed portion coupled to the support portion, an elastically extendable first intermediate portion and a first free end couplable, optionally releasably, to the support portion or another portion of the first strap to form a first loop; and a second strap having a second fixed portion coupled to the support portion, an elastically extendable second intermediate portion and a second free end couplable, optionally releasably, to the support portion or another portion of the second strap to form a second loop.

The first and second loops each define a corresponding first and second channel therethrough, wherein when the straps are not elastically extended and the first and second channels are generally parallel to one another, the first and second channels are laterally offset from one another; wherein the first and second straps are elastically deformable. The first and/or second straps may be configured, such as be elastically deformable or otherwise moveable (for example pivotable as disclosed herein), to define a curved or convoluted pathway extending longitudinally across the support portion through the first and the second loops.

The straps may be positioned such that, in use each first channel may extend generally continuously to each adjacent second channel, when a flexible conduit is secured to the device in use. Alternatively, a gap may be present between adjacent first and second channels, along the curved or convoluted pathway.

The position of the channels can be determined by appropriate positioning of the straps in relation to the support portion, positioning and/or by way of the shape or profile of the strap along its length.

The tertiary attachment may provide an attachment point that is fixed in relation to the support portion. The tertiary attachment may for example comprise a resilient clip, such as a c-clip, for clipping around a tertiary portion of the conduit. The tertiary attachment may comprise a strap (elastic or non-elastic) for securing over or around a conduit.

The securement device may comprise two or more first straps and/or two or more second straps. The or each first strap may be disposed on the support portion with respect to the or each second strap such that the said first and second straps attachable alternately to longitudinally adjacent regions of a flexible conduit. That is to say, no two first regions are adjacent one another, and no two second regions are adjacent one another.

The tertiary attachment may be a further first or second strap. Thus, the tertiary portion of the conduit may be a further first or a further second region of the conduit.

Accordingly, the conduit securement device may comprise two elastically extendable first straps coupled to the support portion; a portion of each first strap being configured to be attached around a corresponding first region of a conduit; and an elastically extendable second strap coupled to the support portion; a portion of the second strap being configured to be attached around a second region of said conduit that is between and longitudinally adjacent to the said first regions. The first and second straps are thereby disposed on the support portion such that the said first and second straps attachable alternately to longitudinally adjacent first and second regions of a flexible conduit.

The conduit securement device may comprise two or more first straps and/or two or more second straps, disposed on the support portion such that the said first and second straps are attachable alternately to longitudinally adjacent first and second regions of a flexible conduit.

By alternately, we mean that along a length of (i.e. longitudinally along) a conduit secured in the device, a region of the conduit is in use attached to a first, followed by a second followed by a first strap, or a second followed by a first followed by a second strap. For example, in some embodiments, in use the curved or convoluted pathway of the flexible conduit may extend alternately through first and second loops.

One or more said straps may be pivotably coupled to the support portion (i.e. about an axis that extends generally perpendicular to the support portion). Thus, when a flexible conduit is secured to the securement device in use, the straps may pivot to maintain the respective first or second forces in a direction normal to the conduit—as might be beneficial for example in a clinical setting where a particularly wide range of motion of the conduit might be expected.

The fixed portions of two first straps and/or two second straps may be coupled to a common region of the support portion. The fixed portions of two first straps and/or two second straps may diverge from a common point. Straps which diverge in this way may have fixings associated with their free ends that are oriented generally perpendicular to a length of the divergent straps, for example to create loops that (when no tension is applied) are at an angle to one another. In use, attaching such diverging straps may assist in urging the flexible conduit into a curved or convoluted pathway and apply a more even force against the conduit in use.

For example, the fixed portions of two first straps and/or two second straps may be pivotable around a common point. The fixed portions of two first straps and/or two second straps may extend from a common point or region, and diverge from one another away from their fixed portions.

In some embodiments two second straps may in use pivot in opposite directions to be attachable around second portions of a flexible conduit to either side of the first portion, longitudinally along the conduit.

The securement device may comprise straps wherein the fixed portions of two or more said straps are longitudinally spaced apart on the support portion. For example, the fixed portions of adjacent first and second straps may be both longitudinally and laterally spaced apart on the support portion.

The securement device may comprise two or more first straps and/or two or more second straps, wherein the free ends of two said first straps, or two said second straps are coupleable to a common point or region of the support portion.

The securement device may comprise two or more first straps and/or two or more second straps, wherein the free ends of two said first straps, or two said second straps are coupleable to laterally spaced apart points or regions of the support portion.

In some embodiments, two first straps or two second straps may extend (optionally pivotally) from fixed portions coupled to a common point or region of the support portion, and comprise free ends coupleable to laterally spaced apart points or regions of the support portion.

The free ends of two or more said first straps, or two or more said second straps may be connected or connectable to one another. For example, this may facilitate the attachment of two or more straps simultaneously.

One or more said straps may be releasably coupled or couplable to the support portion. Such strap(s) may thereby be replaceable. Releasably coupled straps (i.e. at the fixed ends) provide for a single device to be compatible with a range of different applications. For example straps with different lengths, widths, elasticity etc may be selected for a particular purpose, such as the number of flexible conduits, how flexible the conduits are, how easily crushed or crimped a conduit is, how many conduits are to be secured etc.

The free end of a said strap may be releasably coupled to another portion of the strap or the support portion.

The free end of a said strap may be coupled via any suitable fixing, a portion of which is provided on the free end and a complimentary portion of which is provided on another portion of the strap or the support portion. For example, the fixing may comprise a popper, a button, a hook and eyelet, a buckle, a ratchet, a hook and loop fastening (such as a Velcro™ type fastening).

The fixing may comprise a deformable coupling formation, such as an aperture or a deformable ridge or ridges, which can be deformed or stretched through, over or around a fixing formation, such as over/around a button, hook or through a slot; wherein the free end comprises one of the deformable coupling formation or the fixing formation, and another portion of the strap or the support portion comprises the other of the deformable coupling formation or the fixing formation.

A releasable attachment at a fixed or free end of a said strap may be reinforced (in comparison to another portion or portions of the strap, such as an intermediate portion, a loop etc). A releasable attachment may for example be reinforced by overmoulding of a stiffer material defining an aperture, and/or by way of a thickened region of the strap in the vicinity of the releasable attachment(s). Such reinforcement may facilitate elastic extension of the strap whilst limiting or preventing deformation of the releasable attachment that might otherwise cause unintentional release.

In some embodiments, the shore D hardness (i.e. of elastic indentation hardness, measured in accordance with the ASTM D2240-00 testing standard) of a releasable attachment, such as an aperture in a fixed or free end of a strap) is in the range of 10 to 100, 20 to 80 or 40 to 60. The shore D hardness around a releasable attachment may be about 10, 20, 30, 40, 50, 60, 80 or 100.

The shore D hardness of other portions, such as at least an intermediate portion, of a strap may be lower, for example about 75%, 60% or 50% of the shore D hardness around said releasable attachment.

A said strap may be adjustable, so as to adjust the length of the strap, the position of attachment of a free end, the amount of tension applied to a flexible conduit in use, etc.

For example, the free end of a said strap may be provided with a component of a hook and loop fastening, in use releasably coupled to a complimentary portion of hook and loop fastening disposed on the strap or support portion.

A said strap may be adjusted by changing the length along the strap between the fixed portion and free end.

For example, a said strap may comprise a buckle arrangement by which adjustment may be achieved. A strap may comprise an array of apertures spaced apart along a length of the strap, adapted to be positioned over a button or other protrusion extending from the support portion or another portion of the strap. A strap may comprise a ratchet arrangement, such as an array of ribs across the strap (e.g. each extending across a width of the strap, and arrayed along a length of the strap, typically from a free end thereof), and another portion of the strap or the support portion may comprise a slot adapted to receive the ribbed free end or fixed end and engage with one or more said ribs. The invention is not limited to any particular type of strap adjustment and still further alternatives such as a cam lock to pinch or clamp the strap or other alternatives as known to one skilled in the art are also contemplated.

In some clinical settings, it may be desirable for strap tension or maximum and/or minimum strap tension to be preselected, and for a single point of coupling of the free end of a said strap to be provided. For example, this may prevent over tensioning of a conduit that might in the case of a fluid conduit otherwise lead to flattening or blockage. A single point of coupling of the free end may furthermore also be all that is necessary in some settings.

In some embodiments a strap may be cut to length in order to preselect strap tension.

The support portion is configured to in use resist the first and second forces applied by the straps when a longitudinal force is applied to the conduit. This may, in use, prevent or reduce the tendency of the fixed ends of the straps from moving in relation to one another by deformation of the support portion in use.

This may be achieved in a number of ways.

The support portion may be stiffer, or less elastic than the straps.

At least an intermediate portion of the first and/or second strap may be elastically extendable and (for example formed from an elastic material, such as an elastomer or an elastic fabric), and the support portion may comprise for example a thicker material, or a different material, to the elastically extendable intermediate portion of the strap.

The support portion, or at least a part thereof, may be rigid or semi-rigid. By semi rigid we mean that the support portion may be flexible to some degree, to conform to a patient's body part (e.g. for comfort), but still have sufficient rigidity to resist the first and second forces applied by the straps in use. The support portion may for example comprise or be formed from a sheet of plastics material, such as polypropylene, wherein the support portion is sufficiently flexible to be curved around a portion of a patient's limb or other body part when the device is attached thereto, but which is substantially undeformed under the action of the lateral forces applied between the straps, in use.

The device may further comprise an external attachment, for attaching the device to a patient or structure. The external attachment may for example comprise a band or strap for attachment around a patient's limb. The external attachment may comprise a garment, or garment portion, such as a vest, undergarment or limb covering. The external attachment may comprise an adhesive portion, for example in the form of a tape or an adhesive patch or strip, which can be applied to a patient's skin or clothing, or to a structure such as a piece of furniture or a trolley. The external attachment may comprise a releasable fastening, such as a clip or a component of a hook and loop fastening.

An external attachment for attaching the device to a patient may comprise an adjustable band or strap. Adjustability may be provided by any suitable means, such as hook and loop fastenings, buckles, ratchets, laces or the like.

The support portion may be attached to (optionally releasably) or at least partially embedded in or surrounded by the external attachment. For example, the external attachment may comprise a plastic or elastomeric material and the support portion may be a stiffer material such as a polypropylene sheet material of suitable thickness, at least partially embedded in or surrounded by the plastics/elastomeric material.

The support portion may extend to or be formed integrally with the external attachment, whereby when secured to a patient or structure in use, the first and second forces are transmitted to the patient or structure and thereby resisted.

For example, the support portion may be defined by first and second parts of an external attachment, such as the ends of a band. At least one first strap may be coupled to the first part of the support portion, such as the first end of a band; and at least one second strap may be coupled to the second part of the support portion, such as the second end of the band. In use, when the band is attached around a patient's body part or a structure and tightened, tension in the straps is transmitted to the band. Indeed, in some embodiments, portions of the end of a band may function as a said part of the support portion and as a said strap.

In some embodiments, an external attachment such as attaching or tightening a band, is achieved by tensioning of a strap and attachment of the free end of a said first or second strap wrapped around a flexible conduit, to the corresponding first or second part of the support portion.

The support portion may be connected to the external attachment, for example via one or more clips or loops or the like.

The support portion may be removable from the external attachment, for example for cleaning. The support portion and external attachment may be integral, for example formed from a single material.

The material selection for the securement device or component parts thereof may be determined according to the clinical setting. It will be understood by the skilled person that in some circumstances, such as for surgical or emergency applications, the device may be adapted for single use. Whereas for other applications, for example where use of the device may be required over a longer period, material selection for patient comfort, cleaning of the device and/or sterilisation and re-usability of the device may be desirable.

In some embodiments the securement device or one or more component parts thereof is conveniently made from a wipe-clean material. A wipe clean material is a material having a substantially non-porous surface. Examples include plastics materials or elastomeric materials, such as silicones, closed cell neoprenes, polyurethanes, polysterenes, polyolefins and other such materials known by the skilled person for use in medical applications. The securement device may comprise cleanable or sterilizable fabrics, such as plastics or elastomer coated fabrics.

Similarly, the degree of elasticity of the straps may be selected according to a particular purpose. The skilled person will understand that a strap is required to be sufficiently resilient to urge a given conduit or group of conduits into a curved or convoluted pathway, or resist the tendency of a conduit/conduits to straighten; whilst sufficiently elastically extendable to extend in response to typical forces acting between the conduit and the securement device in use. For example, for securement of IV lines, it is desirable for the securement device to be able to isolate the IV line entry point to the vein from at least dropping a fluid bag. Such bags (e.g. of saline, plasma, etc) typically contain around 1 litre of aqueous fluid and so the straps desirably elastically extend but retain an IV line in a curved or convoluted pathway when a longitudinal force of around 1 to 10 N, or 1 to 5 N, or around 1 or 1.5 to 3 N is applied between the device and a retained IV line. The required deflection of a strap to straighten the pathway will in turn depend on the size and configuration of the securement device, but may be between around 5 to 100 mm, 5 to 50 mm, or around 10 to 50 mm or 10 to 25 mm. The material thickness or resilience may be selected accordingly.

In some embodiments, the securement device, or on or more component parts thereof is made from a recyclable material, such as a thermoplastics material, or a biodegradeable material, such as a biodegradable plastics material. Biodegradable plastics materials, such as a cellulosic or other plastics materials based upon plant-based polymers or resins are known in the art.

One or more components of the securement device may be moulded (that is to say formed by moulding, such as compression or injection moulding or the like). The straps may comprise or be formed from a moulded plastics or elastics material. The straps may comprise a fabric, which may be a woven or a non-woven fabric, which fabric may be elastic. A strap comprising a fabric may be coated, for example with a plastics or an elastomeric material.

The support portion may be similarly formed, or optionally overmoulded.

The straps may be attached to the support portion, and optionally the support portion to the external attachment, by any suitable means. The straps, support portion and optionally the external attachment may be integrally formed.

The straps, and optionally the external attachment (such as a band) may be overmoulded over the support portion, or at least a part thereof. Such construction may assist in reducing cavities or seams in which pathogens can collect or grow.

The straps, support portion and optionally the external attachment may be of unitary construction (i.e. moulded, machined or otherwise made from a single piece of material).

The securement device or component parts thereof may be provided with an antipathogenic (such as antibacterial, antifungal or antimicrobial) coating, or may be impregnated with an antipathogenic component. Many such antipathogenic components are known in the art, including metal colloidal compositions, such as silver or copper based, quaternary ammonium compositions or polymers, biguanide polymers and many more.

The securement device or component parts thereof may be provided with a luminescent coating or may be impregnated with a luminescent pigment or dye. This may assist for example in locating the securement device at night or in low light conditions, or may serve as a reminder to a patient that they are still wearing the securement device.

The securement device may comprise two or more groups of first and second straps, each defining in use a curved or convoluted pathway as disclosed herein. Each group of first/second straps may be coupled to a common support portion, or different support portions.

In a further aspect of the invention there is provided a medical system comprising a flexible conduit secured to a securement device:
wherein the securement device comprises:
   a support portion; and
   an elastically extendable first strap, an elastically extendable second strap and a tertiary attachment, coupled to the support portion;
wherein a portion of the first strap is attached around a first region of the conduit; a portion of the second strap is attached around a second region of the conduit longitudinally adjacent to the first region; and the tertiary attachment is attached to a tertiary region of the conduit longitudinally adjacent to the second region; and the flexible conduit is urged into a curved or convoluted pathway;
wherein, when a longitudinal force is applied to the conduit, the first strap and/or the second strap elastically extend or further extend and increase the static friction with the conduit.

The first and/or second straps may be elastically extended when the conduit is in a relaxed configuration. The first and/or second straps may, in some embodiments not be elastically extended when the conduit is in a relaxed configuration, but become elastically extended when a longitudinal force is applied to the conduit.

When elastically extended, the first strap applies a first force to the flexible conduit. When elastically extended, the second strap applies a second force to the flexible conduit.

When the first and the second strap are elastically extended at least a component of the second force may oppose the first force. At least a component of the first and second forces are applied normal to the longitudinal direction of the conduit, in opposite directions in relation to a longitudinal direction of the conduit. At least a component of the second force may oppose the first force. At least a component of the second force may oppose the first force, in relation to a longitudinal direction of the conduit.

The tertiary attachment may provide an attachment point that is fixed in relation to the support portion. The tertiary attachment may for example comprise a resilient clip, such as a c-clip, clipped to or around a tertiary portion of the conduit. The tertiary attachment may comprise a strap (elastic or non-elastic) secured around the tertiary region of the conduit.

The securement device may comprise two or more first straps and/or two or more second straps. The or each first strap may be disposed on the support portion with respect to the or each second strap such that the said first and second straps are attached alternately to longitudinally adjacent regions of a flexible conduit.

The tertiary attachment may be a further first or second strap.

Accordingly, the medical system may comprise a conduit securement device having two elastically extendable first straps coupled to the support portion; wherein a portion of each first strap is attached around a corresponding first region of the conduit; and an elastically extendable second strap coupled to the support portion; wherein a portion of the second strap is attached around a second region of said conduit that is between and longitudinally adjacent to the said first regions. The first and second straps are thereby disposed on the support portion such that the said first and second straps are attached alternately to longitudinally adjacent first and second regions of the flexible conduit.

The conduit securement device may comprise two or more first straps and/or two or more second straps, disposed on the support portion such that the said first and second straps are attached alternately to longitudinally adjacent first and second regions of the flexible conduit.

The conduit may be a fluid line, such as an IV line, a venous line, an arterial line, a drain or the like. The conduit may be a gas line, for example to deliver anaesthetic or oxygen. The, or each, conduit may be an electrical or optical conduit, such as might extend from monitoring devices applied externally or transcutaneously to a patient (e.g. compartment monitors, electrical or optical sensors and the like).

The portions of the first and second straps attached around the conduit may be attached entirely around a conduit, for example forming a corresponding a first or second loop defining a closed first or second channel. The first and second loops may be elastically deformable.

The first and/or second strap may be wrapped around the conduit, for example in opposite directions and/or senses. In embodiments comprising multiple first and/or second straps, as disclosed herein, alternating first and second straps may be wrapped around the conduit in alternating directions or senses.

The portions of the first and second straps attached around the conduit may attach at least partly around a conduit, with means defining an open channel, configured to cup or grip a conduit.

The portions of the first and second straps attached around the conduit may be flexible or elastically deformable and may be flexed or curved to conform to the curved or convoluted pathway.

In some embodiments, the securement device comprises a first strap having a first fixed portion coupled to the support portion, an elastically extended or extendable first intermediate portion and a first free end coupled, optionally releasably, to the support portion or another portion of the first strap to form a first loop around a first region of the conduit; and a second strap having a second fixed portion coupled to the support portion, an elastically extended or extendable second intermediate portion and a second free end coupled, optionally releasably, to the support portion or another portion of the second strap to form a second loop around a longitudinally adjacent second region of the conduit; wherein first and second loops each define a corresponding first and second channel therethrough, and wherein the conduit extends along the first and the second channel through the first and second loops.

The straps may be positioned such that, the or each first channel extends generally continuously to each adjacent second channel.

The securement device of the medical system may further comprise an external attachment.

The external device may be attached or attachable to a patient or structure, for example around a patient's limp, head, neck or torso, or to or around a trolley, bed frame or the like.

The medical system may comprise two or more conduits (of the same or different types or diameters) secured to the securement device. Two or more conduits may be secured along generally the same curved or convoluted pathway. Two or more conduits may extend along a said convoluted pathway generally parallel to one another.

Further alternative or optional features of the securement device correspond to those of other aspects or embodiments of the securement device disclosed herein.

By curved, such as curved pathway, we refer to a pathway including at least one bend. A curved pathway may include one or more than one radius of curvature. By convoluted, such as convoluted pathway, we refer to a pathway including at least two bends in generally opposite but not necessarily co-planar directions along a length of the pathway. A convoluted pathway can be generally serpentine or s-shaped, for example.

It will be understood that the terms lateral, laterally and longitudinal, longitudinally are relative terms defined with respect to the general orientation of a conduit, including a flexible conduit secured to the securement device in use. These terms do not imply any relative magnitude of a longitudinal or lateral direction, or any specific relationship to a length, width or orientation any part of the securement device disclosed herein.

In another aspect there is provided a method of securing a flexible conduit comprising:
attaching an elastically extendable first strap around a region of the flexible conduit;
attaching an elastically extendable second strap around a longitudinally adjacent region of the flexible conduit;
attaching a tertiary attachment to a tertiary region of the flexible conduit; and
urging the flexible conduit into a curved or convoluted pathway defined by the attachments between the conduit and the first strap. second strap and the tertiary attachment.

It will be understood that the steps of the method may be conducted in any suitable order.

In some embodiments, the method may comprise applying a first force to the first region of the flexible conduit by elastically extending the first strap; and/or
applying a second force to the second region of the flexible conduit by elastically extending the second strap.

When the first and the second strap are elastically extended, at least a component of the second force may oppose the first force.

The method may comprise urging the flexible conduit into the curved or convoluted pathway under the action of the first and/or second forces.

In some embodiments, the first strap and/or second strap is elastically extended by the action of attaching the said strap. In some embodiments, the first strap and/or second strap is elastically extended by the action of elastically extending the other of the first strap or second strap, for example by attaching the other said strap. Furthermore, the flexible conduit may be urged to the curved or convoluted pathway during the process of attaching or elastically extending the or each strap. The conduit may be urged to the curved/convoluted pathway from a generally straight configuration, or from another curved or convoluted configuration.

At least a portion of, or all of, the first and second strap may be elastically extendable, as disclosed herein in relation to other aspects.

The method may comprise wrapping the first and/or second strap around the conduit.

The method may comprise wrapping adjacent first and second loops around adjacent first and second regions of the conduit in opposite directions, and optionally opposite senses.

The method may comprise releasably attaching the first strap and/or the second strap to the conduit.

The method may comprise attaching two or more first straps, and/or two or more second straps. The method may comprise attaching the said two or more first and/or two or more second straps to the flexible conduit, whereby corresponding and adjacent first and second regions of the flexible conduit are alternately attached.

The tertiary attachment may be a further first or second strap.

The method may comprise attaching two or more first straps and/or two or more second straps simultaneously. The method may comprise connecting, or using a connection between, two or more said straps together to facilitate simultaneous attachment around the conduit.

The method may comprise releasably coupling a first and/or a second strap around the conduit.

The method may comprise adjusting one or more said straps, by releasing and re-establishing an adjustable releasable fastening. Adjusting may comprise adjusting the length of a said strap (as between a fixed portion and attachment to the conduit). Adjusting may comprise adjusting the curvature of the curved or convoluted pathway. Adjusting may comprise adjusting the tension of a said strap, by elastically extending or further extending at least a portion thereof.

The method may comprise fixing a first strap and/or a second strap to a support portion of a securement device. The method may comprise cutting a first and/or a second strap to a desired length. This may be conducted before, or after securing the conduit.

The method may comprise attaching the external attachment to a patient or structure.

The external attachment may comprise a band or garment, and the method may comprise wrapping the band around a patient's limb or other body part, or placing a patient's limb or body part through or into the external attachment. By body part we refer to a patient's limb (such as a wrist, lower or upper arm, ankle, thigh), waist, chest, head or any other body part to which a device is attached in a clinical setting, as required by a particular circumstance.

The external attachment may comprise an adhesive tape, and the method may comprise placing the securement device on a patient or a garment, and applying the adhesive tape over a part of the securement device. The external attachment may comprise an adhesive surface and the method may comprise preparing or exposing the adhesive surface and applying it to a patient.

The external attachment may comprise a clip or a clamp, and the method may comprise attaching said clip or clamp to a patient's clothing, bed frame or the like. Once such example is a spring biased clip or peg analogous to the external attachment of the HEDclip™ (https://www.he-dk.com/hedclip).

The method may comprise attaching the external attachment before and/or after securing a conduit to the securement device.

In some embodiments, wherein the first and second straps are fixed to respective first and second parts of a support portion of a securement device, and the support portion is defined by first and second parts of an external attachment, such as the ends of a band; attaching the external attachment may comprise the step of attaching the first and/or second strap to the conduit and/or the step of applying the first and/or second force. For example in some embodiments, where the external attachment is a band, the method may comprise wrapping the band around a patient's body part, optionally with the conduit attached around one of the first and/or second strap, and then attaching the other of the first or second strap around the conduit to attach or tighten a band and apply the first and second forces.

The method may comprise securing a conduit to the securement device disclosed herein, and the securement device may comprise any of the features thereof disclosed in relation to the aspects and embodiments thereof. The method may comprise forming a medical system as disclosed herein.

It will be understood that the method may comprise securing two or more conduits to the securement device. Two or more conduits may be secured at the same time. In some embodiments, one or more conduits may be attached at a different time to one or more other conduits. For example a clinician may in some circumstances return to a patient and secure and/or remove conduits—e.g. to replace an IV drip.

The method may comprise removing a conduit from the securement device. Removal may be effected by reversing the securement methods disclosed herein. Removal may be effected by breaking one or more said straps, for example by cutting one or more straps to release a conduit or conduits.

It will be understood that further and optional features of each aspect of the invention correspond to further and optional features of any other aspect of the invention. The methods disclosed herein may for example comprise use of any of the features of the securement device or medical system disclosed herein; whereas the securement device and medical system may include any apparatus required for performance of the method.

### DESCRIPTION OF THE DRAWINGS

Example embodiments will now be described with reference to the following figures in which:

FIG. 1 shows a medical system with two conduits secured to a securement device;

FIG. 2 shows the system of FIG. 1, in cross section along the conduits;

FIGS. 3 and 4 show component parts of the securement device of FIGS. 1 and 2;

FIG. 5 shows the assembled securement device with the straps thereof positioned to receive a conduit;

FIG. 6 shows another medical system disposed around a patient's wrist, with an alternative securement device and conduits secured thereto;

FIGS. 7 and 8 show a further securement device having pivotally attached straps;

FIG. 8A shows the securement device of FIG. 8 attached around a limb;

FIG. 9 shows another securement device with pivotally attached straps, having two straps extending from a common point;

FIG. 10 shows a still further securement device with pivotally attached straps, having two straps extending from a common point, and attached at their free ends to longitudinally spaced apart regions of the support portion of the device; and

FIGS. 11-14 show further securement devices having straps joined by a bridge, and a band defining a part of the support portion and a strap.

FIG. 15 shows another securement device, having straps joined by a bridge.

FIG. 16 shows a securement system with a device having straps diverging from a common point.

FIG. 17 shows the straps and support portion of the securement device of FIG. 16.

### DETAILED DESCRIPTION OF EXAMPLE EMBODIMENTS

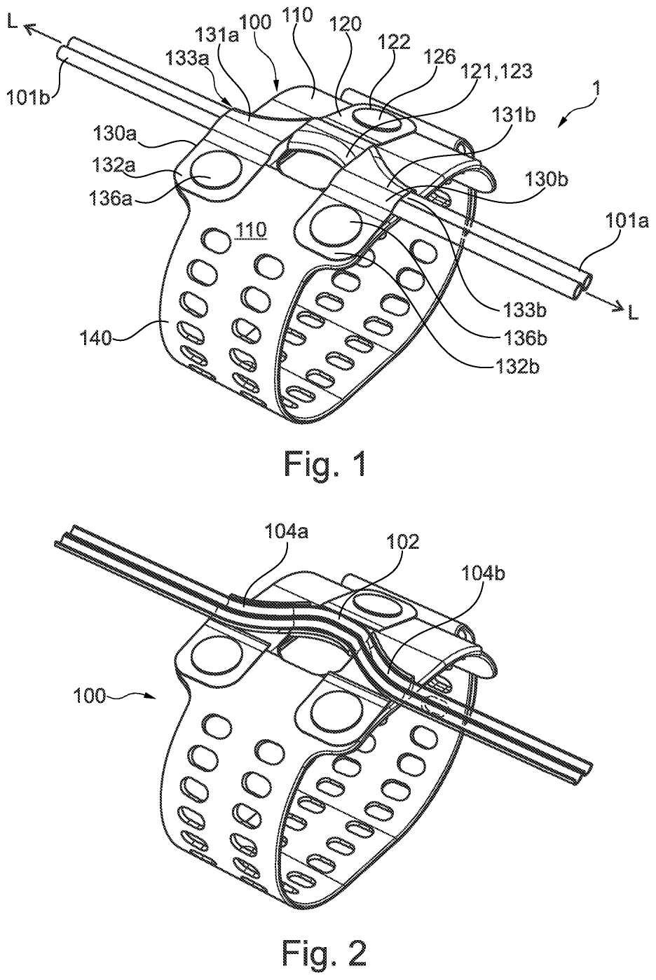

FIG. 1 shows a medical system 1 comprising a securement device 100 and, in the embodiment shown, two flexible conduits 101a and 101b secured to secure securement device 100. FIG. 2 shows the medical system 1 with a cross section view taken longitudinally through the conduits 101a and 101b.

The securement device 100 includes a support portion, indicated generally as 110, an elastically extendable first strap 130a and an elastically extendable second strap 120. In the embodiment shown the securement device 100 comprises two elastically extendable first straps 130a and 130b.

A portion 131a of the first strap 130a is attached around a first region 104a of the conduits (marked in FIG. 2) and a portion 121 of the second strap 120 is attached around a longitudinally adjacent second region 102 of the conduits. A portion 131b of the other first strap 130b is attached around a further first region 104b of the conduits longitudinally adjacent to the second region 102. Accordingly the conduits are attached alternately to the first strap 130a, second strap 120, and the other first strap 130b, along their length.

When the first and second straps are attached around the longitudinally adjacent first and second regions of the flexible conduits 101a, 101b, the conduits are urged into a convoluted pathway. It will be understood that in alternative embodiments a securement device may comprise a different number of straps and the conduit may be urged into alternative curved or convoluted pathways.

In an alternative embodiment (not shown), a tertiary attachment is provided in place of one of the first straps. The tertiary attachment, such as a c-clip, provides a fixed attachment for the conduit, and the conduit in use is urged into a curved or convoluted pathway by the remaining straps.

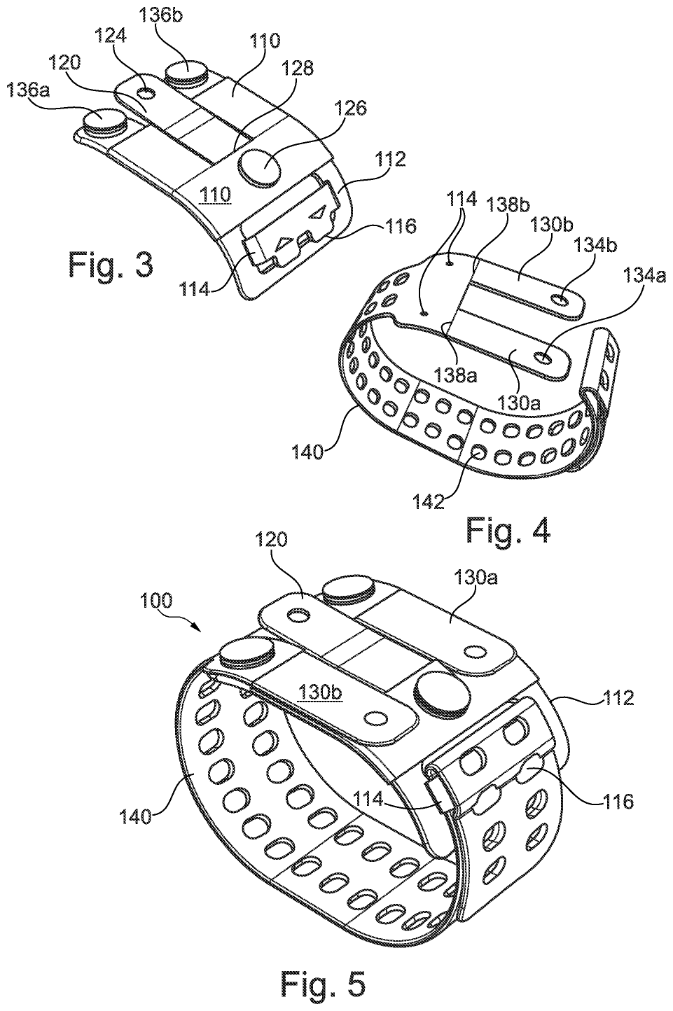

In the embodiment shown the straps are formed from an elastomeric silicone material, and are each releasably attached at their respective free ends 122, 132a, 132b to the support portion 110. Releasable attachment is effected by way of apertures 132, 134a, 134b through the free ends 122, 132a, 132b (see FIG. 3) through which buttons 126, 136a, 136b are introduced. The buttons and apertures together form releasable fixings. It will be understood that in alternative embodiments of the fixings including adjustable fixings may be used.

The first and second straps each have fixed ends 128, 138a, 138b coupled to the support portion 110, as shown in FIGS. 3 and 4.

The second strap is wrapped around the conduits to form a second loop 123, and each of the first straps are wrapped around the conduit in an opposite sense to form corresponding first loops 133a and 133b. The loops each define corresponding first and second channels, which in the embodiment shown extended generally continuously from one another, together defining, at least in part, the convoluted pathway.

The securement device 100 is configured such that when the loops 123, 133a, 133b are formed and the apertures 132, 134a,b and buttons 126, 136a,b attached together in the absence of any conduit extending through the channels, such that the straps are not elastically extended, the first and second channels are laterally offset from one another (i.e. in a direction generally perpendicular to the direction of the loops and channels). Accordingly, when the straps 120, 130a, 130b are attached around the conduit or conduits, as shown in FIGS. 1 and 2, the intermediate portions of the straps defining the loops 123, 133a, 133b are elastically extended. The second strap 120 applies a second force and the first straps 130a, 130b each apply a generally opposed first force to the conduits 101a, 101b to urge them into the convoluted pathway.

When a force is applied between the securement device 100 and portions of the conduits 101a, 101b proximate or distal to the securement device 100, at least a component of which acts longitudinally (direction L) along the conduits, this acts to straighten the convoluted pathway and further elastically extend the first and second straps 120, 130a, 130b, thereby increasing the static friction between these straps and the conduits and resisting longitudinal displacement of the conduits in relation to the securement device. Clinically sensitive areas, in particular the entry point to patient, can thereby be isolated from such forces applied to the conduits.

The inherent elasticity of the straps 120, 130a, 130b also enables the system 1 to accommodate forces applied in other directions between the securement device 100 and the conduits 101a, 101b. In the embodiment shown, the loops 123, 133a, 133b are not attached to the support portion 110 so are able to lift away therefrom and still further reduce the risk of folding or kinking the conduits where they extend out of the second channels 133a, 133b. In use with fluid lines, such as IV lines 101a, 101b, the elasticity of the straps 120, 130a, 130b or the loops 123, 133a, 133b thereof, can be selected such that the straps will elastically extend or deform at lower forces than are required to compress the fluid line.

The securement device 100 further includes an external attachment, in the embodiment shown an adjustable wrist band 140 that extends from the support portion 110.

The curved or convoluted pathway of aspects and embodiments disclosed herein isolates the conduit extending from either side of the securement device from longitudinal or other forces applied to the conduit extending from the other side. A symmetrical curved or convoluted pathway may be of further benefit in this regard. Accordingly, the securement device can be attached either way around, for example to either arm or wrist of a patient.

The construction of the securement device 100 is shown in more detail in FIGS. 3, 4 and 5. FIG. 3 shows the support structure 110, which includes a semi-rigid polypropylene core 112 that integrates the buttons 126, 136a, 136b. The polypropylene is sufficiently rigid to maintain the relative positions of the buttons, and thus the fixed ends 128, 138a, 138b of the straps in use; whist having a degree of flex for patient comfort.

The core 112 also form a buckle 114, with tabs 116 to latch to holes 142 in the adjustable wrist band 140.

The core 112 is overmoulded with an elastomeric silicone material, which also forms the first strap 120. The silicone material has a smooth non-porous surface and can be washed or wiped clean.

As shown in FIG. 4, the band 140 and the first straps 130a, 130b are formed together as a separate moulding, from the silicone material. Apertures 114 are positioned to be secured over the buttons 136a, 136b to fix the ends of the straps 130a, 130b to the support portion 110. The assembled securement device 100 is shown in FIG. 5. To secure a conduit or conduits thereto, a conduit is positioned across the device, the straps wrapped around the conduit and the buttons 126, 136a, 136 passed through the apertures 124, 134a, 134b in the free ends 122, 132a, 132b of the straps 120, 130a, 130b.

Attachment of the straps can be conducted in any order (and indeed before, or after, the wrist band is secured around a patient's wrist), but in the embodiment shown, when one of the first or second bands has been attached, the bands are elastically extended by the action of attaching the other of the first or second bands, so as to urge the conduit into a convoluted pathway as shown in FIG. 1.

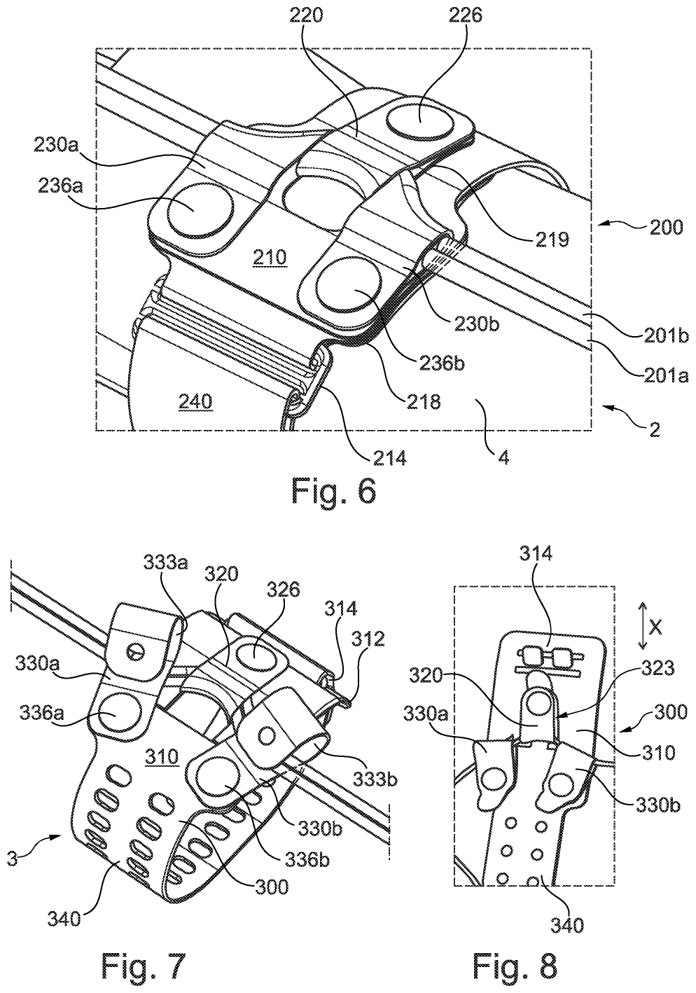

FIG. 6 shows another embodiment of a medical system 2, comprising a securement device 200 around a patient's wrist 4 and conduits 201a, 201b secured to the securement device 200. Features of the securement device in common with the securement device 100 are provided with like reference numerals, incremented by 100.

The securement device 200 is of analogous configuration to the securement device 100, however the wrist band 240 is secured to a buckle 214 comprising two loops; attached to the support portion 210 and the strap 240, respectively. The support portion 210 includes a polypropylene core (not visible in the figure) which includes buttons 226, 236a, 236b on an outer side. A silicone moulding 218 is wrapped around the core, through the buckle 214 and sealed to the core and band 240. A further silicone moulding 219 defines the straps 220, 230a, 230b and is attached to the core over the moulding 218 via the buttons 226, 236a, 236b.

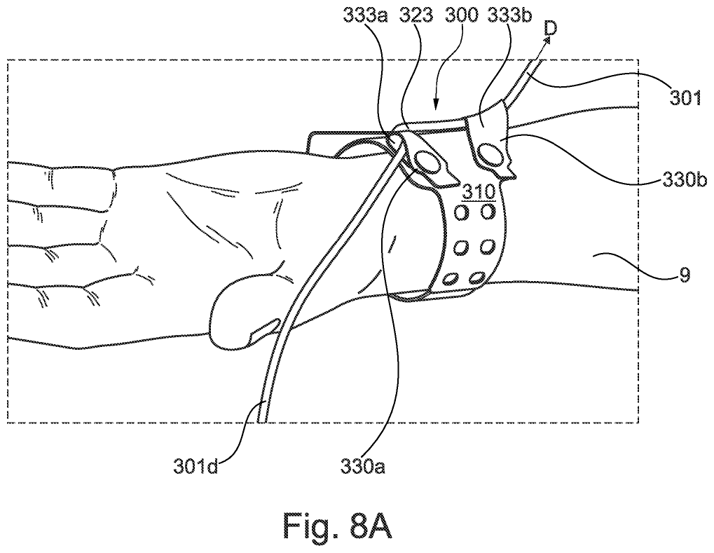

Another embodiment of a securement device 300 and system 3 is shown in FIGS. 7 and 8. Features of the securement device 300 in common with the securement device 100 are provided with like reference numerals, incremented by 200.

The securement device 300 comprises a core 312 overmoulded with a silicone coating to form the support portion 310. A silicone moulding defining the band 340 is attached to the support portion over the buttons 333a, 333b generally as described above for the device 100, with reference to FIGS. 3-5. As discussed above, the band is adjustable securable around a patient's wrist by a buckle 314.

Each of the first strap straps 330a, 330b and second strap 320 are removably attachable at their fixed and free ends to the buttons 326, 336a, 336b. This provides for replacement or selection of the straps, for example in case of breakage or to select a strap having a suitable elasticity for a particular purpose.

The straps 320, 330a, 330b are pivotally mounted to the buttons 326, 336a, 336b, and so can pivot to accommodate relatively extreme movement of a conduit 301 extending through the device, as might be encountered in certain situations, such as for relatively mobile or ambulant patients.

When a conduit 301 or conduits 301a, 301b is/are secured to the device 300, as illustrated in FIGS. 7 and 8, the straps are wrapped around the conduit(s) to form loops 323, 333a, 333b that define corresponding channels (in this embodiment, moveable to be spaced apart from one another along the conduit) that urges the conduit(s) into a curved or a convoluted pathway. When the device 300 is in a relaxed configuration (as shown in FIG. 8) then the some or all of the straps need not be elastically extended. As with the securement devices 100, 200, when the straps are not elastically extended 220, 230a, 230 and oriented around the buttons so that the channels through the loops are parallel, the channels are laterally offset (direction X) from one another. Thus, in use, when a force is applied to the conduit having at least a longitudinal component, the straps elastically extend as the curved or convoluted pathway begins to straighten, and the opposed first and second forces applied between the elastically elongated straps increase friction between the straps and the conduit(s) and resists longitudinal displacement of the conduit(s). Similarly, since the loops 333a, 333b are not attached to the support portion 310, large out of plane movements between the conduit and the securement device 300, including movements that lead to elastic extension or deformation of the straps, can be accommodated.

FIG. 8A shows the securement device 300 of FIGS. 7 and 8 in use around a model of a patient's arm. Tension is being applied to the conduit in a direction “out of plane” from the longitudinal direction of the conduit and convoluted pathway, generally upwardly in the orientation shown in the figure, with respect to the support portion 310. The loops 333a and 333b (and also 323) of the straps 330a, 330b and 320 are free to move in relation to the support portion. Thus, and as illustrated in the figure, in use such relatively large movement of the conduit with respect to the securement device in a wide range of directions can thereby be accommodated, as the distance and position of the loops (i.e. the portions of the straps attached to the conduit) in relation to the support portion can be varied. This arrangement further reduces the risk of crimping or folding the conduit 301 as it exits the channel defined by the respective loop.

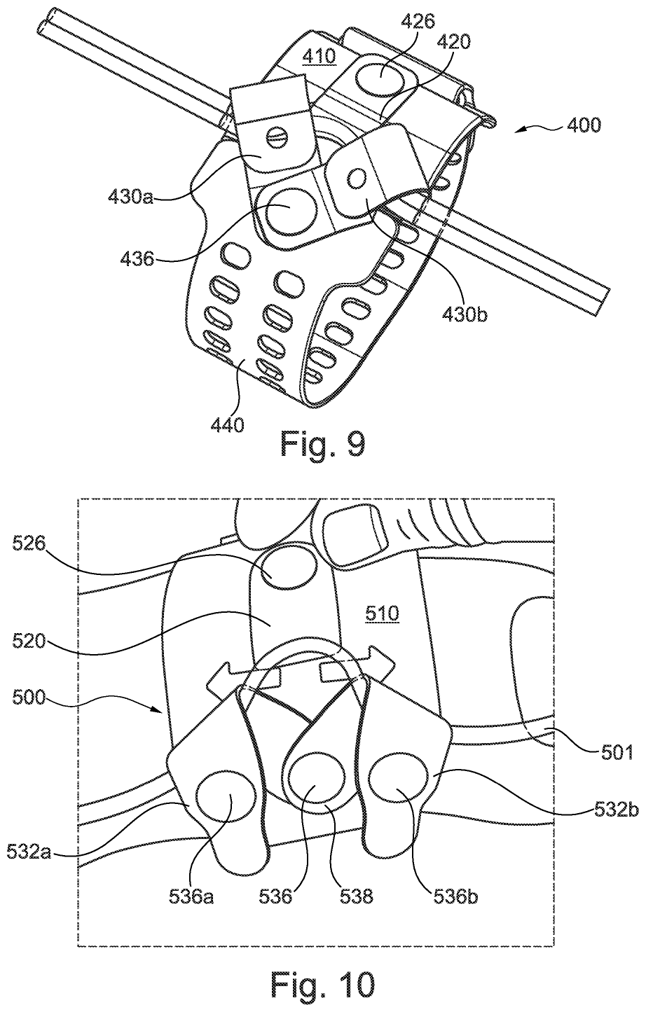

Another securement device 400 is shown in FIG. 9. The securement device 400 is constructed in an analogous manner to the device 300, and features in common therewith are provided with like reference numerals, incremented by 100.

The securement device 400 differs from the securement device 300 in that the two first straps 430a, 430b are pivotally (and releasably) attached to a common point of the support portion, over button 436. The securement device 500 shown in FIG. 10 is analogous to the securement device 400, and like features are provided with like reference numerals incremented by 100. The releasably attached fixed ends 538 of the first straps are attached over a central button 536. The first straps 536a, 536b are wrapped around a conduit 501 and attached at their free ends 532a, 532b to longitudinally spaced apart buttons 533a, 533b, together with the second strap 520, defining a curved or convoluted pathway of the conduit 501 across the device 500. The diverging first straps assist in urging the conduit into a curved or convoluted pathway, and apply an even pressure through the loops thereof.

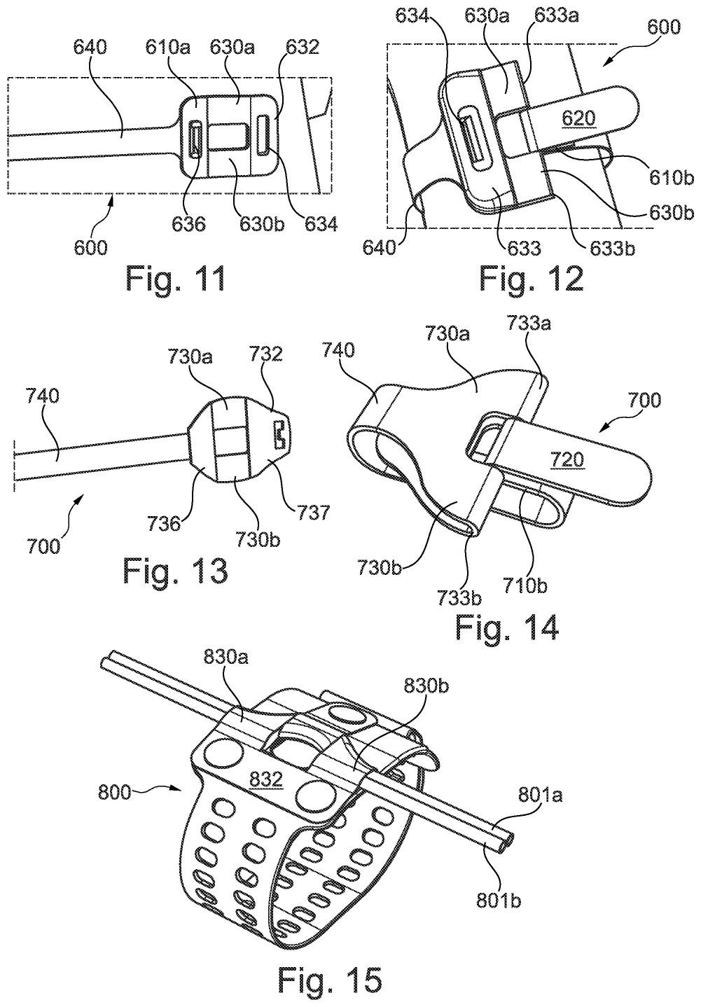

FIGS. 11-14 show alternative embodiments 600, 700 of a securement device. The devices 600, 700 each have a support structure having a first part 610a, 710a. Secured thereto are first straps 630a, 630b and 730a, 730b. The free ends of the first straps are joined to one another across their free ends, by a bridge 632, 732 such that the first straps can be attached at the same time as one another, for convenience.

The support portions and bridges include complimentary parts of a fixing by which the first straps are attached to the support portion, around a conduit, in use.

The securement device 600 comprises a catch 636, which is introduced through a slot 634 in the bridge 632 (as shown in FIG. 12) to secure the straps 630a, 630b. As can be seen in FIG. 12, the free ends of the first straps and the bridge 632 are bonded to a polypropylene reinforcement 633 to provide for a “snap-fit” over the catch 636. The intermediate portions of the second straps 630a, 630b, which define loops 633a, 633b in use, are formed from the elastically extendable silicone material. As discussed above, in alternative embodiments, other materials may be used.

The securement device 700 comprises a surface 736 of the support part 710a formed as a hook component of a hook and loop fastening. The opposed surface 737 of the bridge 732 is coated with the complimentary loop material of a hook and loop fastening, such that the first straps 730a, 730b can be adjustably and releasably attached to the support part 710a.

Also bonded to the support parts 610a, 710a (and co-moulded from the silicone material together with the first straps) is a band 640, 740. In use, the distal end of the bands 640, 740 are looped around a conduit (omitted from the figures for clarity) between the first straps 630a, 630b and 730a, 730b and releasably secured to the band.

Regions of the end of the band therefore function as another part 610b, 710b of the support portion and as a first band 620, 720.

The details of a releasable and adjustable fastening of the end of the bands 640, 740 defining the second straps 620, 720 are omitted for clarity, but any suitable fastening, such as a hook and loop fastening, or pegs/openings as shown with respect to other embodiments, may be used.

In use, the once wrapped around a conduit generally in the configurations illustrated in FIGS. 12 and 14 second straps 620, 720 and thus the wrist bands 640, 740 are tightened to elastically extend the respective first and second straps and urge a conduit into a convoluted pathway.

FIG. 15 shows a medical system 8 comprising a securement device 800 and conduits 801a, 801b secured thereto. The securement device 800 is analogous to the securement device 100, except that the first straps 830a, 830b are joined to one another at their free ends, by a bridge 832.

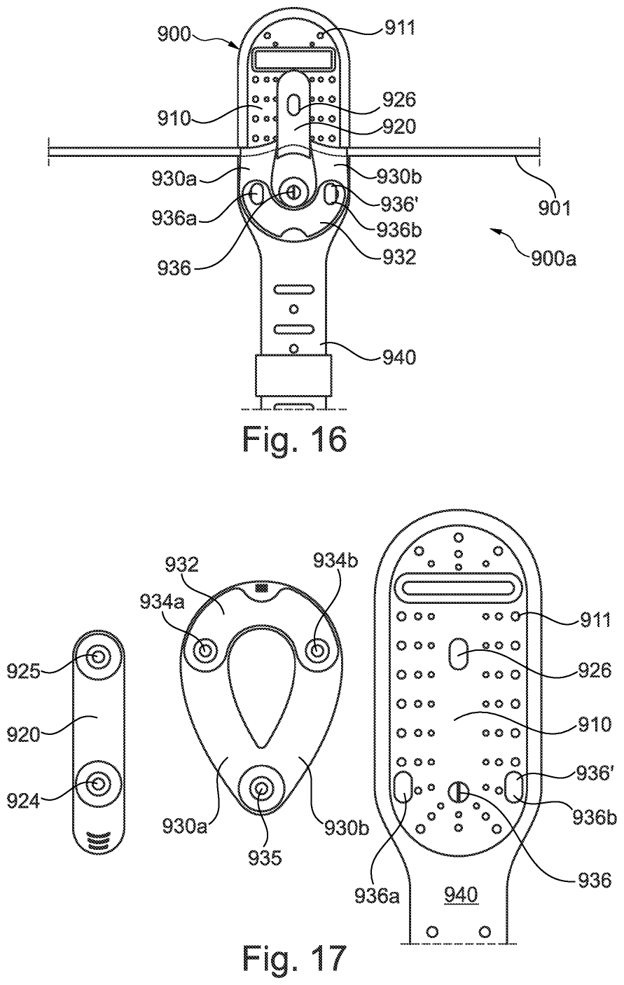

FIG. 16 shows another securement system 900a comprising a device 900 and a conduit 901 secured thereto, which is secured to the straps thereof and extends across the device 900 along a convoluted pathway. The securement device 900 is constructed in an analogous manner to the device 100, and features in common therewith are provided with like reference numerals, incremented by 800.

The two first straps 930a, 930b (and releasably) attached to a common point of the support portion over button 936, and diverge therefrom. The diverging first straps 930a, 930b assist in urging the conduit into a curved or convoluted pathway, and apply an even pressure through the loops thereof.

FIG. 17 shows two first straps 930a, 930b for use with the device 900 joined at their free ends by a bridge 932. The straps diverge from aperture 932, by which the straps are attached to the button 926 on the support portion 910. Also shown in FIG. 17 is the second strap 920 and the support portion 910 and band 940. The support portion 910 in the embodiment shown is ventilated with perforations 911. The apertures 924, 925, 934a, 934b and 935 by which the fixed and free ends of the straps are attached to the buttons 926, 936a, 936b are overmoulded over a stiffer material, to provide additional strength. The bridge is also reinforced and overmoulded in the embodiment shown. The buttons 926, 936a, 936b are asymmetrical, having an extended overhanging portion (such as the portion 936′ marked on the figure) to assist in secure attachment of the straps.

Whilst the invention has been described in connection with the foregoing illustrative embodiments, various modifications, additions and alterations may be made to the invention by one skilled in the art without departing from the scope of the claims.

1. A conduit securement device, for securing a flexible conduit, the device comprising:
a support portion;
an elastically extendable first strap coupled to the support portion; a portion of the first strap being configured to be attached around a first region of a conduit;
an elastically extendable second strap coupled to the support portion; a portion of the second strap being configured to be attached around a longitudinally adjacent second region of said conduit; and
a tertiary attachment for attaching a further region of said flexible conduit to the support portion said further region being longitudinally adjacent the second region;
wherein, when the first and second straps are attached around longitudinally adjacent first and second regions of a flexible conduit and secure the flexible conduit to the securement device, the flexible conduit is urged into a curved or convoluted pathway by the straps
wherein, tension applied longitudinally to a conduit secured to the device acts to straighten the curved or convoluted pathway and extend or further extend the first and/or second strap and increase a static friction with the conduit.

2. The securement device of claim 1, wherein the first strap and the second strap are configured to wrap around a conduit, in opposite directions or senses.

3. (canceled)

4. The securement device of claim 1, wherein, when a longitudinal force is applied along the conduit proximal to or distal to the securement device, the first strap elastically extends or further extends and applies a first force to the flexible conduit; and/or the second strap elastically extends or further extends and applies a second force to the flexible conduit;
wherein at least a component of the first and/or second force is applied normal to the longitudinal direction of the conduit.

5. The securement device of claim 1, wherein the securement device comprises a first strap having a first fixed portion coupled to the support portion, an elastically extendable first intermediate portion and a first free end couplable to the support portion or another portion of the first strap to form a first loop; and
a second strap having a second fixed portion coupled to the support portion, an elastically extendable second intermediate portion and a second free end couplable to the support portion or another portion of the second strap to form a second loop;
first and second loops each defining a corresponding first and second channel therethrough;
wherein, when the straps are not elastically extended and the first and second channels are generally parallel to one another, the first and second channels are laterally offset from one another; and
wherein the first and second straps configured to define a curved or convoluted pathway extending longitudinally across the support portion through the first and the second loops.

6. The securement device of claim 5, wherein the straps are positioned such that, in use the first channel extends generally continuously to the adjacent second channel, when a flexible conduit is secured to the device.

7. The securement device of claim 1, wherein the tertiary attachment is a further first strap attachable around a further first portion of a conduit longitudinally adjacent the second portion.

8. (canceled)

9. The securement device of claim 7, comprising two elastically extendable first straps coupled to the support portion; a portion of each first strap being configured to be attached around a corresponding first region of a conduit; and an elastically extendable second strap coupled to the support portion; a portion of the second strap being configured to be attached around a second region of said conduit that is between and longitudinally adjacent to the said first regions.

10. (canceled)

11. The securement device of claim 1, wherein one or more said straps is adjustable.

12. The securement device of claim 1, further comprising an external attachment, for attaching the device to a patient, wherein external attachment comprises a band for attachment around a patient's limb, or wherein the external attachment comprises a garment, or garment portion.

13. The securement device of claim 1, wherein the securement device, or one or more parts thereof, is made from a wipe-clean material having a substantially non-porous surface; and/or wherein the securement device or one or more component parts thereof is provided with an antipathogenic coating, or impregnated with an antipathogenic component; and/or wherein the securement device or one or more component parts thereof is provided with a luminescent coating or impregnated with a luminescent pigment or dye.

14. (canceled)

15. A medical system comprising a flexible conduit secured to a securement device:
wherein the securement device comprises:
a support portion; and
an elastically extendable first strap, an elastically extendable second strap and a tertiary attachment, coupled to the support portion;
wherein a portion of the first strap is attached around a first region of the conduit; a portion of the second strap is attached around a second region of the conduit longitudinally adjacent to the first region; and the tertiary attachment is attached to a tertiary region of the conduit longitudinally adjacent to the second region; and the flexible conduit is urged into a curved or convoluted pathway;
wherein, when a longitudinal force is applied to the conduit, the first strap and/or the second strap elastically extend or further extend and increase the static friction with the conduit.

16. The medical system of claim 15, wherein the flexible conduit is a fluid line, such or an electrical or optical conduit.

17. (canceled)

18. The medical system of claim 15, comprising two or more conduits of the same or different types or diameters secured to the securement device along the curved or convoluted pathway generally parallel to one another.

19. A method of securing a flexible conduit comprising:
attaching a first elastically extendable strap around a region of the flexible conduit;
attaching a second elastically extendable strap around a longitudinally adjacent region of the flexible conduit;
attaching a tertiary attachment to a tertiary region of the flexible conduit; and
urging the flexible conduit into a curved or convoluted pathway defined by the attachments between the conduit and the first and second straps.

20. The method of claim 19, comprising applying a first force to the first region of the flexible conduit by elastically extending the first strap; and/or
applying a second force to the second region of the flexible conduit by elastically extending the second strap.

21. The method of claim 20 wherein, when the first and second straps are elastically extended, at least a component of the second force opposes the first force.

22. (canceled)

23. The method of claim 19, comprising wrapping the first and/or second strap around the conduit, in opposite directions or senses.

24. The method of claim 19, comprising attaching two or more first and/or two or more second straps to the flexible conduit, whereby corresponding and adjacent first and second regions of the flexible conduit are alternately attached.

25. The method of claim 19, wherein the straps form part of a securement device, wherein the securement device comprises an external attachment, wherein the method comprises attaching the external attachment to a patient or structure before or after securing the flexible conduit.

26. The method of claim 19, comprising securing two or more flexible conduits to the securement device or the same or different types.
